(19) United States
(12) Patent Application Publication
(10) Pub. No.: US 2026/0157669 A1
(43) Pub. Date: Jun. 11, 2026

(54) NON-INVASIVE WEARABLE SYSTEM AND METHOD FOR DETECTING BUBBLES IN A BLOODSTREAM AND/OR TISSUE

(71) Applicant: Vivonics, Inc., Bedford, MA (US)

(72) Inventor: Michele Pierro, Westford, MA (US)

(21) Appl. No.: 19/308,446

(22) Filed: Aug. 25, 2025

Related U.S. Application Data
(60) Provisional application No. 63/686,991, filed on Aug. 26, 2024.

(51) Int. Cl. A61B 5/1455; A61B 5/00; A61B 5/053

(52) U.S. Cl. CPC A61B 5/14552; A61B 5/053; A61B 5/6801

(57) ABSTRACT

A non-invasive wearable system for detecting bubbles in a bloodstream and/or tissue includes at least one first emitter configured to emit light at one or more first predetermined wavelengths sensitive to arterial blood into the bloodstream and tissue of a human subject. At least one second emitter is configured to emit light at one or more second predetermined wavelengths sensitive to venous blood into the bloodstream and the tissue of a human subject. At least one detector is configured to detect the light emitted at the one or more first predetermined wavelengths and output first photoplethysmography (PPG) waveforms and detect the light emitted at the one or more second predetermined wavelengths and output second PPG waveforms. A processing subsystem is coupled to the at least one first emitter, the at least one second emitter, and the at least one detector. The processing subsystem is configured to calculate arterial oxygen saturation (SaO₂) from the first PPG waveforms and calculate venous oxygen saturation (SvO₂) from the second PPG waveforms and detect changes in the SaO₂ and the SvO₂ to detect the presence of gas bubbles in at least one of the blood stream or the tissue of the human subject.

### RELATED APPLICATIONS

This application claims benefit of and priority to U.S. Provisional Application Ser. No. 63/686,991 filed Aug. 26, 2024, under 35 U.S.C. § § 119, 120, 363, 365, and 37 C.F.R. § 1.55 and § 1.78, which is incorporated herein by this reference.

### FIELD OF THE INVENTION

This invention relates to a non-invasive wearable system and method for detecting bubbles in a bloodstream and/or tissue and in one example to a non-invasive wearable system and method thereof for detecting bubbles in a bloodstream to monitor and detect decompression sickness (DCS).

### BACKGROUND OF THE INVENTION

Bubbles within the blood may cause vascular obstruction, blood flow disruption, and pattern changes. In the most severe situations, bubbles in the blood may lead to hypoxic tissue damage. Decompression sickness (DCS) occurs when dissolved gases, primarily nitrogen, come out of solution and form bubbles in the body at atmospheric pressure, or partial pressure of dissolved gas, decreases. DCS can lead to various symptoms ranging from joint pain and dizziness to more severe neurological issues. DCS may be considered a leading risk and time-limiting factor for diving operations. Despite the fact that diving operational guidelines are widely available and are typically designed for the majority of divers covering a range of diving conditions, DCS symptoms and outcomes vary between individuals. Therefore, a more tailored approach is needed for DCS detection and monitoring which may benefit individual diver physical conditions. Additionally, DCS is a significant concern for NASA astronauts due to the unique environmental conditions of space. In the microgravity environment of space, body fluids, including blood, shift upwards, which can change the way gases are dissolved and distributed in tissues. Additionally, when astronauts transition from the pressurized cabin of a spacecraft to the lower pressure or varying gas mixture of a space suit for extra vehicular activities (EVA) such as a spacewalk or travel on a planet surface, the rapid change in pressure can lead to DCS. Pre-breathing the gas mixture of the space suit before exiting the spacecraft (which may have a different gas mixture or partial pressures) can help avoid bubble formation upon exit but is time consuming.

The increased pressure gradient between inspired gas and the dissolved gas in the body at depth results in an equilibration of the differential pressure leading to saturation. See Lang, M. A., et al., Diving physiology and decompression sickness: Considerations from humans and marine animals, Research and Discoveries: The Revolution of Science Through Scuba, 2013, incorporated by reference herein. The composition of the air we breathe consists primarily of nitrogen (about 79%), an inert gas that is absorbed and dissolved in the bloodstream and tissues. See Lang, M. A., et al., cited supra and W. Fraser, Relation between complement activation and susceptibility to decompression sickness, Journal of Applied Physiology, 1987, 62(3): p. 1160-1166, incorporated by reference herein. The nitrogen partial pressure at sea level in the lungs and surrounding tissues is in equilibrium. Ambient pressure increases on descent, and it causes denser air in the lungs to be driven into the tissues to maintain equilibrium (“ongassing”). On ascent, the ambient pressure decreases, leading to an increase in the nitrogen partial pressure in the tissues to be driven into the lungs (“offgassing”). It takes time for nitrogen to enter and leave the body. While ascending, the body begins the nitrogen elimination process. However, if too much nitrogen is still present while surfacing or after surfacing, the excess nitrogen forms bubbles in the body, creating a microscopic clot that impairs circulation and can lead to serious damage to the endothelial lining of the vessels. See Lang, M. A., et al. cited supra and Nossum et al., Endothelial damage by bubbles in the pulmonary artery of the pig, Undersea & hyperbaric medicine, 1999, 26(1): p. 1, incorporated by reference herein. This condition is known as Decompression Sickness (DCS) or “the Bends” and its symptoms range from skin rash, extreme fatigue, coughing, and painful joints to paralysis and unconsciousness.

Gas bubbles in liquid are strong reflectors of sound; thus, various modes of ultrasound have been used for detection of circulating vascular gas bubbles. Conventional doppler systems are often used. However, their cumbersome form factor, need for highly trained operators, and long acquisition times make this solution less than ideal candidate for continuous monitoring of gas bubbles during diving operations. Unfortunately, ultrasound-based approaches are not directly translated into wearable solutions. Thus, these ultrasound-based approaches may be confined to post diving assessments and analysis. This may result in undetected DCS episodes that occur underwater, and which could be prevented if continuous air bubble monitoring was available. Real-time gas bubble detection may provide insights about what dive profiles lead to bubble formation which could be used to provide new dive tables, computer algorithms, and may provide information to astronauts in the unique environmental conditions of space.

### SUMMARY OF THE INVENTION

In one aspect, a non-invasive wearable system for detecting bubbles in a bloodstream and/or tissue is featured. The system includes at least one first emitter configured to emit light at one or more first predetermined wavelengths sensitive to arterial blood into the bloodstream and tissue of a human subject. At least one second emitter is configured to emit light at one or more second predetermined wavelengths sensitive to venous blood into the bloodstream and the tissue of a human subject. At least one detector is configured to detect the light emitted at the one or more first predetermined wavelengths and output first photoplethysmography (PPG) waveforms and detect the light emitted at the one or more second predetermined wavelengths and output second PPG waveforms. A processing subsystem is coupled to at least one first emitter, the at least one second emitter, and the at least one detector. The processing subsystem is configured to determine an indication of arterial oxygen saturation (SaO₂) from the first PPG waveforms and determine an indication of venous oxygen saturation (SvO₂) from the second PPG waveforms and detect changes in the SaO₂ and the SvO₂ to detect the presence of gas bubbles in at least one of the blood stream or the tissue of the human subject.

In one embodiment, the processing subsystem may determine changes in the SaO₂ to determine the amount and size of the gas bubbles in the arterial blood. The processing subsystem may determine changes in the SvO₂ to determine the amount and size of the gas bubbles in the venous blood. The at least one detector may be configured to detect reflected or transmitted light emitted at the one or more first predetermined wavelengths and the one or more second predetermined wavelengths. The one or more first predetermined wavelengths may absorb light associated with oxygenated hemoglobin and the one or more second predetermined wavelengths may absorb light associated with deoxygenated hemoglobin. The system may include an electrical impedance spectroscopy (EIS) subsystem configured to determine a plurality of electrical impedances of a volume of the tissue at a plurality of different electrical stimulations at a plurality of frequencies and amplitudes to calculate size and quantity of gas bubbles in the volume of tissue. The determination of the gas bubbles in the bloodstream or tissue of the human subject may be combined with the size and quantity of gas bubbles calculated by the EIS subsystem to provide an improved determination of gas bubbles in the blood and/or tissue of the human subject.

In another aspect, a non-invasive wearable method for detecting bubbles in a bloodstream and/or tissue is featured. The method includes emitting light at one or more first predetermined wavelengths sensitive to arterial blood into the bloodstream and tissue of a human subject, emitting light at one or more second predetermined wavelengths sensitive to venous blood into the bloodstream and the tissue of a human subject, detecting the light emitted at the one or more first predetermined wavelengths and outputting first photoplethysmography (PPG) waveforms, detecting the light emitted at the one or more second predetermined wavelengths and outputting second PPG waveforms, determining an indication of arterial oxygen saturation (SaO₂) from the first PPG waveforms, determining an indication venous oxygen saturation (SvO₂) waveforms from the second PPG waveforms, and detecting changes in the SaO₂ and the SvO₂ to detect the presence of gas bubbles in at least one of the bloodstream and/or the tissue of the human subject.

In one embodiment, the method may include determining changes in the SaO₂ to determine the amount and size of the gas bubbles in the arterial blood. The method may include determining changes in the SvO₂ to determine the amount and size of the gas bubbles in the venous blood. The method may include detecting reflected or transmitted light emitted at the one or more first predetermined wavelengths and the one or more second predetermined wavelengths. The one or more first predetermined wavelengths may absorb light associated with oxygenated hemoglobin and the one or more second predetermined wavelengths may absorb light associated with deoxygenated hemoglobin. The method may include determining a plurality of electrical impedances of a volume of the tissue at a plurality of different electrical stimulations at a plurality of frequencies and amplitudes to calculate size and quantity of gas bubbles in the volume of tissue. The determination of the gas bubbles in the bloodstream or tissue of the human subject may be combined with the calculated size and quantity of gas bubbles to provide an improved determination of gas bubbles in the blood or tissue of the human subject.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic diagram showing the primary components of one example of non-invasive wearable system for detecting bubbles in a bloodstream and/or tissue;

FIG. 2 shows an example of the system shown in FIG. 1 integrated with a computer subsystem and including a real-time display of bubble trends (formation) and other relevant parameters during diving or operation by astronauts;

FIG. 3 shows an example of the system shown in FIG. 1 secured to a human subject; and

FIG. 4 is a flow chart showing one example of the primary steps of the method for detecting gas bubbles.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

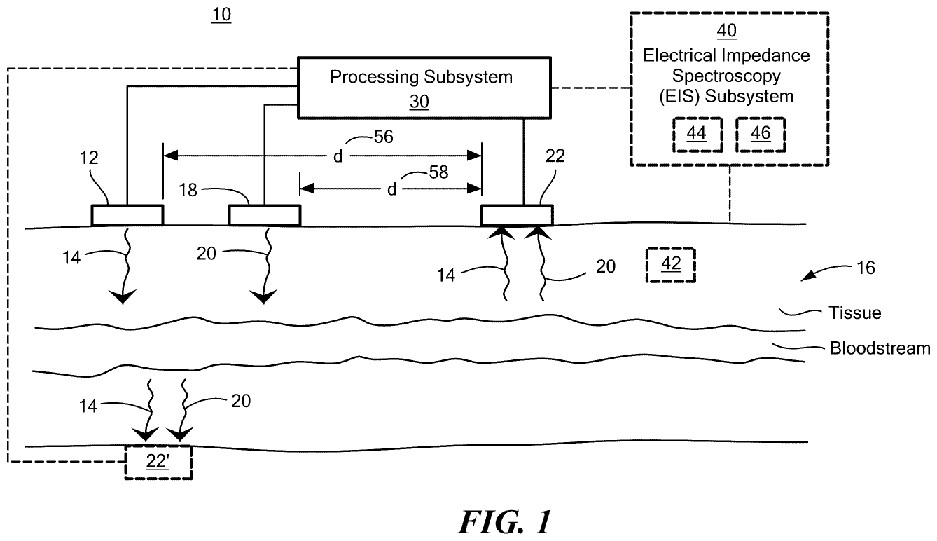

There is shown in FIG. 1, one example of non-invasive wearable system 10 for detecting bubbles in a bloodstream and/or tissue of a human subject. System 10 preferably includes at least one first emitter 12 which emits light 14 at one or more first predetermined wavelengths sensitive to arterial blood into the bloodstream and tissue, exemplarily indicated at 16, of a human subject. In one example, light 14 is preferably in the near infrared or infrared light spectrum and has a wavelength preferably associated with the oxygenated hemoglobin ([HbO]), e.g., about 690 nm, or similar length wavelength. System 10 also preferably includes at least one second emitter 18 which emits light 20 at one or more second predetermined wavelengths sensitive to venous blood into the bloodstream and the tissue 18 of a human subject. In one example, light 20 is preferably in the near infrared or infrared light spectrum and has a wavelength associated with the deoxygenated hemoglobin ([Hb]), e.g., about 830 nm or similar length wavelength.

In one example, first emitter 12 and second emitter 22 may be light emitting diodes (LEDs), laser diodes, or similar type light sources known to those skilled in the art.

System 10 also preferably includes at least one detector 22 which detects light 14 emitted at the one or more first predetermined wavelengths sensitive to arterial blood and outputs first photoplethysmography (PPG) waveforms. At least one detector 22 also preferably detects light 20 emitted at the one or more second predetermined wavelengths sensitive to venous blood and outputs second PPG waveforms. In one example, detector 22 may detect reflected light 14 and light 20 as shown. In another example, at least one detector 22′ may detect light 14 and light 22 that is transmitted through the tissue and bloodstream of the human subject as shown.

System 10 also preferably includes processing subsystem 30 coupled to at least one first emitter 12, the at least one second emitter 18, and the at least one detector 22 and/or at least one detector 22′. Processing subsystem 30 preferably determines an indication of arterial oxygen saturation (SaO₂) from the first PPG waveforms and determines an indication of arterial venous saturation (SvO₂) from the second PPG waveforms and detects changes in the SaO₂ and the SvO₂ to detect the presence of gas bubbles in at least one of the bloodstream or the tissue of the human subject.

In one example, processing subsystem 30 preferably determines changes in the SaO₂ to determine the amount and size of the gas bubbles in the arterial blood. Processing subsystem 30 preferably determines changes in the SvO₂ to determine the amount and size of the gas bubbles in the venous blood.

In one design, system 10 may include electrical impedance spectroscopy (EIS) subsystem 40 which preferably determines a plurality of electrical impedances of a volume of the tissue, exemplarily indicated at 42, at a plurality of different electrical stimulations at a plurality of frequencies and amplitudes to calculate size and quantity of gas bubbles in the volume of tissue. In one example, the peak-to-peak amplitudes may be about 10 to 100 mV, or similar peak-to-peak amplitudes and the frequency may be about 1 kHz to about 1 MHz, or similar frequencies. The tissue electrical impedance determined by EIS subsystem 40 is a function of its structure and is preferably used to differentiate tissue with and without air bubbles.

In one example, the determination of the gas bubbles in the bloodstream and/or tissue of the human subject discussed above using the determined arterial oxygen saturation (SaO₂) from the first PPG waveforms and determined arterial venous saturation (SvO₂) from the second PPG waveform may be combined with the size and quantity of gas bubbles calculated by the EIS subsystem 40 to provide an improved determination of gas bubbles in the blood or tissue of the human subject.

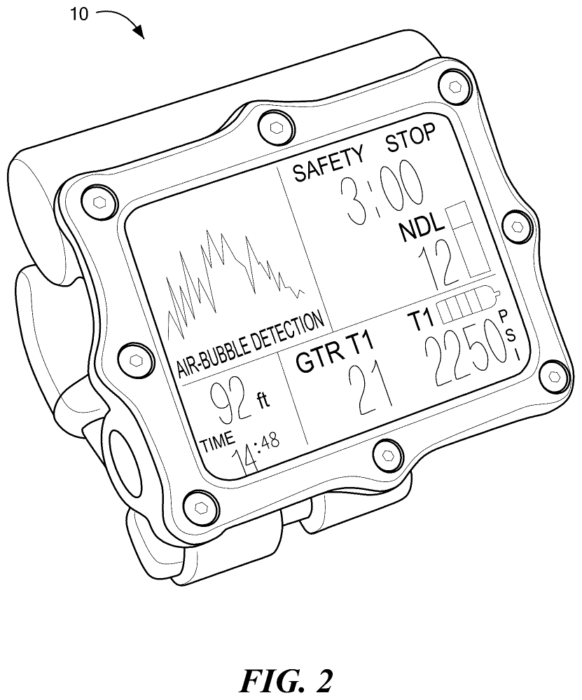

In one design, system 10 may be integrated with a computer subsystem, e.g., as shown in FIG. 2, and preferably includes a real-time display of bubble trends (formation) and other relevant parameters as shown during diving or operation by astronauts.

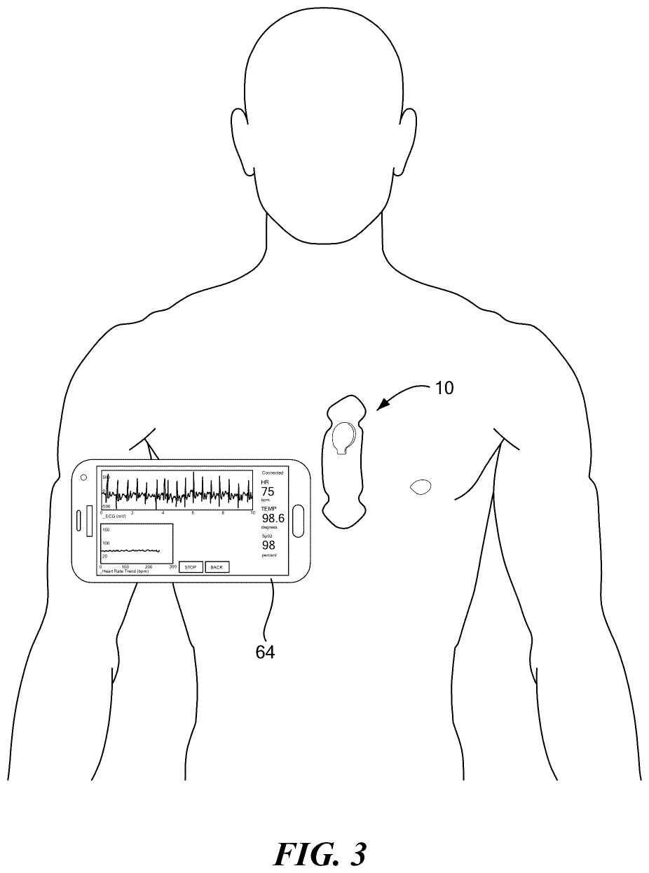

Non-invasive wearable system 10 and method thereof for detecting bubbles in the bloodstream and/or tissue preferably detects gas bubbles, e.g., nitrogen bubbles, helium bubbles, or any gas that can dissolve in blood in the bloodstream and/or in tissue, preferably in real-time. System 10 is preferably lightweight, small, e.g., as shown in FIGS. 2 and 3, and may be comfortably worn by divers or astronauts without posing any additional risk due to pressure points that might trap bubbles. System 10 is preferably easy to apply and position on the skin and preferably requires no user interaction following measurement onset. System 10 may be worn on the wrist, arm, leg or torso of a human subject. In one example, system 10 and preferably combines an optical-based technology discussed above with EIS subsystem 40 to utilize the power of both modalities to detect gas bubbles in the bloodstream and/or in tissue of a living subject with unparalleled ease and accuracy.

The typical waveform pattern as measured by photoplethysmography (PPG) associated with venous or arterial saturation (SvO₂ and SaO₂) as discussed above and blood volume oscillations may be altered by the presence of gas bubbles and these changes can be attributed to the presence and size of circulating inert gases (bubbles).

Thus, by correlating measured changes in SvO₂ and SaO₂ blood volume oscillations using the first and second PPG waveforms discussed above, system 10 and the method thereof preferably quantifies gas bubbles in the blood stream and/or tissue and discriminates their presence among the arterial and venous compartment. Additionally, EIS subsystem 40 may be utilized to provide an improved determination of gas bubbles in the blood or tissue of the human subject.

Processing subsystem 30 preferably correlates measured changes in SaO₂, SvO₂ blood volume oscillations (PPG waveforms) to quantify gas bubbles in the blood stream and/or the tissue and preferably discriminates the presence of gas bubbles among the arterial and venous compartment. Processing subsystem 30 may include one or more processors, an application-specific integrated circuit (ASIC), firmware, hardware, and/or software (including firmware, resident software, micro-code, and the like) or a combination of both hardware and programs that may all generally be referred to herein as a “processing subsystem”.

System 10 and the method thereof preferably non-invasively and continuously, in real time, or near time, detects gas bubbles in the bloodstream and/or tissue to effectively monitor for DCS.

DCS symptoms can lead to excruciating pain and can be lethal to divers. See Lang, M. A., et al., cited supra. When underwater, a diver breathing compressed air out of a tank normally absorbs the air into fatty body tissues instead of exhaling it all out, which is typically safe for humans to do. However, ascending to the surface too fast after a deep dive can cause those gases to expand and form into bubbles inside the body. See Ward, C., D. McCullough, and W. Fraser, Relation between complement activation and susceptibility to decompression sickness, Journal of Applied Physiology, 1987, 62(3): p. 1160-1166; Papadopoulou, V., et al., A critical review of physiological bubble formation in hyperbaric decompression, Advances in colloid and interface science, 2013, 191, p. 22-30, and Barak, M. and Y. Katz, Microbubbles. Chest, 2005, 128(4), p. 2918-2932, all incorporated by reference herein.

While the precise sites for formation of microbubbles after a decompression are still a matter of debate (see Kim, K. J., mDiving and Hyperbaric Medicine. Korean Journal of Anesthesiology, 2008, 54(5), p. 479-485, incorporated by reference herein), bubbles have been found in both the bloodstream and tissue, hence the importance of using a dual approach to measuring both.

Once a bubble, typically made of inert gases such as nitrogen and helium, has entered the bloodstream, if its size is small enough to not directly occlude a vessel, it will be transported by the blood and follow the normal circulation into the right chambers of the heart and the lungs. See Papadopoulou, V., et al., Circulatory bubble dynamics: from physical to biological aspects, Advances in colloid and interface science, 2014, 206, p. 239-249, incorporated by reference herein. The bubble will travel at the velocity of blood provided it is small compared to the vessel cross-sectional area, with velocities from 0.03 cm/s in the capillaries to 40 cm/s in the aorta. See Butler, B. and B. Hills, The lung as a filter for microbubbles. Journal of Applied Physiology, 1979, 47(3), p. 537-543, incorporated by reference herein.

As opposed to inert gasses, metabolic gases such oxygen, bound for the most part to hemoglobin, do not typically cause problems as they are directly used by the body and recycled through breathing. Oxygenated and deoxygenated hemoglobin act as chromophores in the bloodstream, where concentration changes lead to absorption changes to which optical-based methods are sensitive. See Pierro, M. L., et al., Phase-amplitude investigation of spontaneous low-frequency oscillations of cerebral hemodynamics with near-infrared spectroscopy: a sleep study in human subjects, Neuroimage, 2012, 63(3), p. 1571-1584, incorporated by reference herein. This phenomenon takes place when light of specific wavelengths propagates through biological tissues, and the light absorbing molecules (oxygenated and deoxygenated hemoglobin) interrupt the photons'propagation at different levels. See Pierro, M.L., et al., cited supra. By properly choosing one or more first predetermined wavelengths and the one or more second predetermined wavelengths discussed above with reference to FIG. 1, it is possible to maximize the sensitivity of the optical measurements to the change in hemoglobin concentration and use this information as a surrogate of the blood flow. System 10 and the method thereof preferably selects and utilizes two wavelengths, e.g., the one or more first predetermined wavelengths and the one or more second predetermined wavelengths discussed above to determines an indication of or calculate the oxygenated ([HbO]) and deoxygenated ([Hb]) hemoglobin concentration. In one example, the one or more first predetermined wavelengths and the one or more second predetermined wavelengths preferably allow for the detection of oscillations in blood volume due to heartbeats or other physiological processes by measuring the variations in light absorption by blood in the tissue.

The one or more first predetermined wavelengths and the one or more second predetermined wavelengths discussed above are preferably selected as part of the design of system 10 preferably based on known absorptivity spectra (plots of absorbance vs. wavelength). The two wavelengths are preferably chosen for their sensitivity to different chromophores, particularly oxygenated hemoglobin and deoxygenated hemoglobin to determine the blood oxygen saturation levels (SvO2 and SaO2).

The distance, d-56, FIG. 1, between first emitter 12 and shared detector 22 and the distance, d-58, between second emitter 18 and shared detector 22 are preferably selected to effectively provide a penetration distance from which the reflected or scattered light returns. The distance between light emitter or sources 12, 18 and detector 22 is preferably selected as part of the design of system 10 based on knowledge of physiology, i.e., the typical depth of the blood vessels in the region of interest versus the tissue layer above.

In this example, the oxygenated and deoxygenated hemoglobin concentrations are preferably derived by translating absorption coefficients measurements at about 690 nm and about 830 nm into a linear system of two equations. See Fantini, S., A haemodynamic model for the physiological interpretation of in vivo measurements of the concentration and oxygen saturation of haemoglobin, Physics in medicine and biology, 2002, 47(18), p. N249, and Fantini et at., Semi-infinite-geometry boundary problem for light migration in highly scattering media: a frequency-domain study in the diffusion approximation, JOSA B, 1994, 11(10), p. 2128-2138, all incorporated by reference herein. An algorithm (see Pierro, M. L., et al., cited supra and Franceschini, M. A., et al., Near-infrared spiroximetry: noninvasive measurements of venous saturation in piglets and human subjects, Journal of Applied Physiology, 2002, 92(1), p. 372-384, incorporated by reference herein, has been developed to measure oxygen saturation in the venous and arterial compartment (SvO₂ and SaO₂ respectively). In one example, the Modified Beer-Lambert Law (MBLL) may be used to quantify changes in oxyhemoglobin (HbO) and deoxyhemoglobin (Hb) concentrations by accounting for light scattering in biological tissues. Bubbles within the blood cause vascular obstruction, blood flow disruption, and pattern changes, which in the most severe cases can lead to hypoxic tissue damage. See Lang, M. A., et al., cited supra. As a result, the regular waveform patterns (photoplethysmography signals: PPG) associated with SvO₂ and SaO₂ blood volume oscillations is altered by the presence of air bubbles. These changes can be attributed to the presence and size of circulating inert gases (bubbles). Thus, system 10 and the method thereof correlates measured changes in SvO₂ and SaO₂ blood volume oscillations (PPG waveforms) to quantify gas bubbles in the blood stream and/or tissue and discriminates their presence among the arterial and venous compartment to effectively and efficiently detect bubbles in the bloodstream and/or tissue. In one example, system 10 and the method thereof preferably combines the advanced signal features extraction (time and frequency analysis). The blood flow waveform in arterial and venous compartments are preferably estimated from SvO₂ and SaO₂ oscillations and, in one example, may be combined with signals from EIS subsystem 40 from air bubble detection in tissue to provide comprehensive information about an individual diver or astronaut DCS or other physiological conditions.

SvO₂ and SaO₂ are preferably calculated based on the measurements of amplitude oscillations of [HbO] and [Hb] at the spontaneous respiratory and cardiac frequency respectively. See Franceschini, M. A, et al., cited supra,, and Franceschini, M. A., E. Gratton, and S. Fantini, Noninvasive optical method of measuring tissue and arterial saturation: an application to absolute pulse oximetry of the brain. Optics letters, 1999, 24(12), p. 829-831, incorporated by reference herein. These two frequencies (about 0.2-0.4 Hz and about 1 Hz) represent the venous and arterial volume fraction in tissues (see Pierro, cited supra and are preferably used to retrieve their respective oxygen saturation waveforms for blood flow pattern estimation.

While the optical approach of system 10 and the method thereof provides an unprecedented opportunity to measure gas bubbles in blood stream and/or tissue, the novel EIS methodology, in one example preferably provided by EIS subsystem 40, may provide a complementary technique for gas bubbles detection in tissue. Tissue electrical impedance is a function of its structure and it can be used to differentiate normal tissue with and without bubbles. See Da Silva, J. E., J. M. De Sá, and J. Jossinet, Classification of breast tissue by electrical impedance spectroscopy, Medical and Biological Engineering and Computing, 2000, 38(1), p. 26-30, incorporated by reference herein. Specifically, the electrical impedance of a volume of tissue 42, FIG. 1, at a series of different electrical stimulation frequencies, may provide information about the cell population and thus the tissue structure. See Dean, D., et al., Electrical impedance spectroscopy study of biological tissues, Journal of electrostatics, 2008, 66(3-4), p. 165-177, incorporated by reference herein. It is a technique that, as with the optical system 10 and method thereof, may be used by system 10 and the method thereof to detect bubble formation.

In one design, system 10, preferably includes at least one emitter 12, at least one emitter 18, at least one detector 22, at least one processor 30, and integrated electronics to provide for multiplexing capabilities to provide for the first predetermined wavelength provided by first emitter 12, e.g., about 850 nm wavelength or similar length wavelength, and provide the second predetermined wavelength provided by second emitter 18, e.g., about 690 nm wavelength or similar length wavelength. In one prototype design, light emitters 12, 18, and their respective distances, d-56, d-58 from shared detector 22 preferably optimize both venous and arterial compartments sampling. This design preferably facilitates blood flow sensitivity and data acquisition. The algorithm for the assessment of the blood flow pattern preferably uses an algorithm by the inventor hereof, see Pierro, M. nL., et al., cited supra bas₂d on SvO₂ and SaO₂ calculation. Tissue gas bubbles may be detected by bio-impedance measurements using EIS subsystem 40. EIS subsystem 40 preferably includes impedance/gain-phase analyzer 44, which preferably includes at least one microprocessor 46. Microprocessor 46 may be used to provide firmware support for the multiplexing circuit necessary for the correct functioning of the optical apparatus. In one example, stimuli may be preferably provided using small skin-contact electrodes.

System 10 and the method thereof preferably identifies a range of frequency capable of detecting the presence of gas bubbles in the blood stream and/or tissue on a micron scale. The impedance spectrum may be obtained from voltages obtained across a synthetic (in vitro) tissue in response to sinusoidal electric current excitation of known amplitude and phase at different frequencies. In one example, EIS subsystem 40 may include an impedance spectroscopy subsystem, in one example available from Eliko^{®}. (Aiandi 13/1, Tallinn 12918, Estonia), which preferably provides 15-frequency measurements for detecting subtle impedance changes in real-time, e.g., about 1000 samples/sec.

As discussed above, system 10 is preferably a wearable device that may be comfortably worn by divers or astronauts without posing any additional risk due to pressure points that might trap bubbles. System 10 is preferably easy to apply and position on the skin and preferably requires no user interaction following measurement onset. FIG. 3 shows one example of a non-invasive wearable system 10 secured to a living subject. In this example, system 10 preferably wirelessly communicates to electronic device 64, e.g., a smart device, mobile phone, tablet, or similar type device, to detect the presence of bubbles in the bloodstream and/or tissue of a human subject and provide additional physiological parameters, such as heart rate, respiration rate, heart rate variability, resting heart rate, changes in blood pressure, blood oxygen saturation, and the like.

Preferably, system 10 and method detects the presence of gas bubbles in pulsatile flow with bubble sizes down to about 5-10 μm, which can be used to infer about their average size and concentration.

In one design, individual bloodstream and tissue samples may be synchronized via timestamp for post-processing analysis. The timestamp will preferably allow post-processing analysis and investigation of bubble events and measurements, along with time and dive depth as measured by the dive computer.

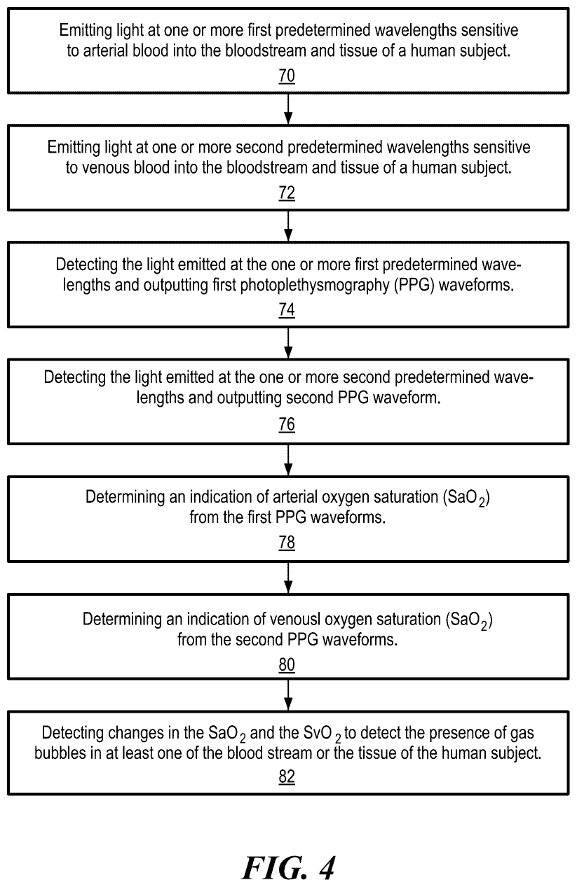

One example of the non-invasive wearable method for detecting bubbles in a bloodstream and/or tissue includes emitting light at one or more first predetermined wavelengths sensitive to arterial blood into the bloodstream and tissue of a human subject, step 70, FIG. 4, emitting light one or more second predetermined wavelengths sensitive to venous blood into the bloodstream and the tissue of a human subject, step 72, detecting the light emitted at the one or more first predetermined wavelengths and outputting first photoplethysmography (PPG) waveforms, step 74. The method also includes detecting the light emitted at the one or more second predetermined wavelengths and output a second PPG waveforms, step 76, calculating arterial oxygen saturation (SaO₂) from the first PPG waveforms, step 78, calculating venous oxygen saturation (SvO₂) waveforms from the second PPG waveforms, step 80, and detecting changes in the SaO₂ and the SvO₂ to detect the presence of gas bubbles in at least one of the blood stream or the tissue of the human subject, step 82.

In one example, the method may include determining changes in the SaO₂ to determine the amount and size of the gas bubbles in the arterial blood. The method may include determining changes in the SvO₂ to determine the amount and size of the gas bubbles in the venous blood. The method may include detecting reflected or transmitted light emitted at the one or more first predetermined wavelengths and the one or more second predetermined wavelengths. The one or more first predetermined wavelengths may absorb light associated with oxygenated hemoglobin and the one or more second predetermined wavelengths absorb light associated with deoxygenated hemoglobin.

The method may include determining a plurality of electrical impedances of a volume of the tissue at a plurality of different electrical stimulations at a plurality of frequencies and amplitudes to calculate size and quantity of gas bubbles in the volume of tissue. The determination of the gas bubbles in the bloodstream or tissue of the human subject may be combined with the calculated size and quantity of gas bubbles to provide an improved determination of gas bubbles in the blood or tissue of the human subject.

The result is system 10 and the method thereof effectively and efficiently detects bubbles in the bloodstream and/or tissue of a living subject and may be used to effectively monitor and detect DCS.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

1. A non-invasive wearable system for detecting bubbles in a bloodstream and/or tissue, the system comprising:
at least one first emitter configured to emit light at one or more first predetermined wavelengths sensitive to arterial blood into the bloodstream and tissue of a human subject;
at least one second emitter configured to emit light at one or more second predetermined wavelengths sensitive to venous blood into the bloodstream and the tissue of a human subject;
at least one detector configured to detect the light emitted at the one or more first predetermined wavelengths and output first photoplethysmography (PPG) waveforms and detect the light emitted at detect light at the one or more second predetermined wavelengths and output second PPG waveforms; and
a processing subsystem coupled to at least one first emitter, the at least one second emitter, and the at least one detector, the processing subsystem configured to calculate arterial oxygen saturation (SaO₂) from the first PPG waveforms and calculate venous oxygen saturation (SvO₂) from the second PPG waveforms and detect changes in the SaO₂ and the SvO₂ to detect the presence of gas bubbles in at least one of the blood stream or the tissue of the human subject.

2. The system of claim 1 in which the processing subsystem determines changes in the SaO₂ to determine the amount and size of the gas bubbles in the arterial blood.

3. The system of claim 1 in which the processing subsystem determines changes in the SvO₂ to determine the amount and size of the gas bubbles in the venous blood.

4. The system of claim 1 in which the at least one detector is configured to detect reflected or transmitted light emitted at the one or more first predetermined wavelengths and the one or more second predetermined wavelengths.

5. The system of claim 1 in which the one or more first predetermined wavelengths absorb light associated with oxygenated hemoglobin and the one or more second predetermined wavelengths absorb light associated with deoxygenated hemoglobin.

6. The system of claim 1 including an electrical impedance spectroscopy (EIS) subsystem configured to determine a plurality of electrical impedances of a volume of the tissue at a plurality of different electrical stimulations at a plurality of frequencies and amplitudes to calculate size and quantity of gas bubbles in the volume of tissue.

7. The system of claim 6 in which the determination of the gas bubbles in the bloodstream or tissue of the human subject is combined with the size and quantity of gas bubbles calculated by the EIS subsystem to provide an improved determination of gas bubbles in the blood or tissue of the human subject.

8. A non-invasive wearable method for detecting bubbles in a bloodstream and/or tissue, the method comprising:
emitting light at one or more first predetermined wavelengths sensitive to arterial blood into the bloodstream and tissue of a human subject;
emitting light at one or more second predetermined wavelengths sensitive to venous blood into the bloodstream and the tissue of a human subject;
detecting the light emitted at the one or more first predetermined wavelengths and outputting first photoplethysmography (PPG) waveforms;
detecting the light emitted at the one or more second predetermined wavelengths and output second PPG waveforms at the one or more second predetermined wavelengths;
calculating arterial oxygen saturation (SaO₂) from the first PPG waveforms;
calculating venous oxygen saturation (SvO₂) waveforms from the second PPG waveforms; and
detecting changes in the SaO₂ and the SvO₂ to detect the presence of gas bubbles in at least one of the blood stream or the tissue of the human subject.

9. The method of claim 8 including determining changes in the SaO₂ to determine the amount and size of the gas bubbles in the arterial blood.

10. The method of claim 8 including determining changes in the SvO₂ to determine the amount and size of the gas bubbles in the venous blood.

11. The method of claim 8 including detecting reflected or transmitted light emitted at the one or more first predetermined wavelengths and the one or more second predetermined wavelengths.

12. The method of claim 8 in which the one or more first predetermined wavelengths absorb light associated with oxygenated hemoglobin and the one or more second predetermined wavelengths absorb light associated with deoxygenated hemoglobin.

13. The method of claim 8 including determining a plurality of electrical impedances of a volume of the tissue at a plurality of different electrical stimulations at a plurality of frequencies and amplitudes to calculate size and quantity of gas bubbles in the volume of tissue.

14. The system of claim 8 in which the determination of the gas bubbles in the bloodstream or tissue of the human subject is combined with the calculated size and quantity of gas bubbles to provide an improved determination of gas bubbles in the blood or tissue of the human subject.
